SEQUENCE LISTING (30 sequences)

SEQ ID NO: 1 - protein, 30 aa, Homo sapiens
MEPAGPAPGRLGPLLCLLLAASCAWSGVAG

SEQ ID NO: 2 - protein, 116 aa, Homo sapiens
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRA

SEQ ID NO: 3 - protein, 343 aa, Homo sapiens
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIY

SEQ ID NO: 4 - protein, 116 aa, Homo sapiens
EEELQVIQPDKSVLVAAGESATLRCTATSLIPVGPIQWFRGAGPGRELIYNQKEGHFPRVTTVSDLTKRNNMDFSIRIGNITPADAGTYYCVKFRKGSPDDVEFKSGAGTELSVRA

SEQ ID NO: 5 - protein, 116 aa, Homo sapiens
EEELQVIQPDKSVLVAAGETATLRCTATSLIPVGPIQWFRGAGPGRELIYNQKEGHFPRVTTVSELTKRNNMDFSIRIGNITPADAGTYYCVKFRKGSPDDVEFKSGAGTELSVRA

SEQ ID NO: 6 - protein, 343 aa, Homo sapiens
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTLTENKDGTYNWMSWLLVNVSAHRDDVKLTCQVEHDGQPAVSKSHDLKVSAHPKEQGSNTAAENTGSNERNIY

SEQ ID NO: 7 - protein, 15 aa, Homo sapiens
EPKSCDKTHTCPPCP

SEQ ID NO: 8 - protein, 15 aa, Homo sapiens
EPKSSDKTHTCPPCP

SEQ ID NO: 9 - protein, 10 aa, Homo sapiens
DKTHTCPPCP

SEQ ID NO: 10 - protein, 10 aa, Homo sapiens
DKTHTSPPSP

SEQ ID NO: 11 - protein, 4 aa, Homo sapiens
GSSG

SEQ ID NO: 12 - protein, 4 aa, Homo sapiens
GSGS

SEQ ID NO: 13 - protein, 217 aa, Homo sapiens
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 363 aa, Homo sapiens
MEPAGPAPGRLGPLLCLLLAASCAWSGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 373 aa, Homo sapiens
MEPAGPAPGRLGPLLCLLLAASCAWSGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRADKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 367 aa, Homo sapiens
MEPAGPAPGRLGPLLCLLLAASCAWSGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAGSGSAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 605 aa, Homo sapiens [sequence omitted]

SEQ ID NO: 18 - protein, 605 aa, Homo sapiens
MEPAGPAPGRLGPLLCLLLAASCAWSGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 363 aa, Homo sapiens
MEPAGPAPGRLGPLLCLLLAASCAWSGVAGEEELQVIQPDKSVLVAAGETATLRCTATSLIPVGPIQWFRGAGPGRELIYNQKEGHFPRVTTVSELTKRNNMDFSIRIGNITPADAGTYYCVKFRKGSPDDVEFKSGAGTELSVRAAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 373 aa, Homo sapiens
MEPAGPAPGRLGPLLCLLLAASCAWSGVAGEEELQVIQPDKSVLVAAGETATLRCTATSLIPVGPIQWFRGAGPGRELIYNQKEGHFPRVTTVSELTKRNNMDFSIRIGNITPADAGTYYCVKFRKGSPDDVEFKSGAGTELSVRADKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 605 aa, Homo sapiens
MEPAGPAPGRLGPLLCLLLAASCAWSGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTLTENKDGTYNWMSWLLVNVSAHRDDVKLTCQVEHDGQPAVSKSHDLKVSAHPKEQGSNTAAENTGSNERNIYEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 600 aa, Homo sapiens
MEPAGPAPGRLGPLLCLLLAASCAWSGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTLTENKDGTYNWMSWLLVNVSAHRDDVKLTCQVEHDGQPAVSKSHDLKVSAHPKEQGSNTAAENTGSNERNIYDKTHTSPPSPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 504 aa, Homo sapiens
MEPAGPAPGRLGPLLCLLLAASCAWSGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYIVVGVVCTLLVALLMAALYLVRIRQKKAQGSTSSTRLHEPEKNAREITQDTNDITYADLNLPKGKKPAPQAAEPNNHTEYASIQTSPQPASEDTLTYADLDMVHLNRTPKQPAPKPEPSFSEYASVQVPRK

SEQ ID NO: 24 - protein, 323 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 25 - DNA, 90 bp, Homo sapiens
atggagcccgccggcccggcccccggccgcctcgggccgctgctctgcctgctgctcgccgcgtcctgcgcctggtcaggagtggcgggt

SEQ ID NO: 26 - DNA, 348 bp, Homo sapiens
gaggaggagctgcaggtgattcagcctgacaagtccgtgttggttgcagctggagagacagccactctgcgctgcactgcgacctctctgatccctgtggggcccatccagtggttcagaggagctggaccaggccgggaattaatctacaatcaaaaagaaggccacttcccccgggtaacaactgtttcagacctcacaaagagaaacaacatggacttttccatccgcatcggtaacatcaccccagcagatgccggcacctactactgtgtgaagttccggaaagggagccccgatgacgtggagtttaagtctggagcaggcactgagctgtctgtgcgcgcc

SEQ ID NO: 27 - DNA, 1029 bp, Homo sapiens
gaggaggagctgcaggtgattcagcctgacaagtccgtgttggttgcagctggagagacagccactctgcgctgcactgcgacctctctgatccctgtggggcccatccagtggttcagaggagctggaccaggccgggaattaatctacaatcaaaaagaaggccacttcccccgggtaacaactgtttcagacctcacaaagagaaacaacatggacttttccatccgcatcggtaacatcaccccagcagatgccggcacctactactgtgtgaagttccggaaagggagccccgatgacgtggagtttaagtctggagcaggcactgagctgtctgtgcgcgccaaaccctctgcccccgtggtatcgggccctgcggcgagggccacacctcagcacacagtgagcttcacctgcgagtcccacggcttctcacccagagacatcaccctgaaatggttcaaaaatgggaatgagctctcagacttccagaccaacgtggaccccgtaggagagagcgtgtcctacagcatccacagcacagccaaggtggtgctgacccgcgaggacgttcactctcaagtcatctgcgaggtggcccacgtcaccttgcagggggaccctcttcgtgggactgccaacttgtctgagaccatccgagttccacccaccttggaggttactcaacagcccgtgagggcagagaaccaggtgaatgtcacctgccaggtgaggaagttctacccccagagactacagctgacctggttggagaatggaaacgtgtcccggacagaaacggcctcaaccgttacagagaacaaggatggtacctacaactggatgagctggctcctggtgaatgtatctgcccacagggatgatgtgaagctcacctgccaggtggagcatgacgggcagccagcggtcagcaaaagccatgacctgaaggtctcagcccacccgaaggagcagggctcaaataccgccgctgagaacactggatctaatgaacggaacatctat

SEQ ID NO: 28 - DNA, 348 bp, Homo sapiens
gaggaggagctgcaggtgattcagcctgacaagtccgtgttggttgcagctggagagtcagccactctgcgctgcactgcgacctctctgatccctgtggggcccatccagtggttcagaggagctggaccaggccgggaattaatctacaatcaaaaagaaggccacttcccccgggtaacaactgtttcagacctcacaaagagaaacaacatggacttttccatccgcatcggtaacatcaccccagcagatgccggcacctactactgtgtgaagttccggaaagggagccccgatgacgtggagtttaagtctggagcaggcactgagctgtctgtgcgcgcc

SEQ ID NO: 29 - DNA, 348 bp, Homo sapiens
gaggaggagctgcaggtgattcagcctgacaagtccgtgttggttgcagctggagagacagccactctgcgctgcactgcgacctctctgatccctgtggggcccatccagtggttcagaggagctggaccaggccgggaattaatctacaatcaaaaagaaggccacttcccccgggtaacaactgtttcagagctcacaaagagaaacaacatggacttttccatccgcatcggtaacatcaccccagcagatgccggcacctactactgtgtgaagttccggaaagggagccccgatgacgtggagtttaagtctggagcaggcactgagctgtctgtgcgcgcc

SEQ ID NO: 30 - DNA, 1029 bp, Homo sapiens
gaggaggagctgcaggtgattcagcctgacaagtccgtgttggttgcagctggagagacagccactctgcgctgcactgcgacctctctgatccctgtggggcccatccagtggttcagaggagctggaccaggccgggaattaatctacaatcaaaaagaaggccacttcccccgggtaacaactgtttcagacctcacaaagagaaacaacatggacttttccatccgcatcggtaacatcaccccagcagatgccggcacctactactgtgtgaagttccggaaagggagccccgatgacgtggagtttaagtctggagcaggcactgagctgtctgtgcgcgccaaaccctctgcccccgtggtatcgggccctgcggcgagggccacacctcagcacacagtgagcttcacctgcgagtcccacggcttctcacccagagacatcaccctgaaatggttcaaaaatgggaatgagctctcagacttccagaccaacgtggaccccgtaggagagagcgtgtcctacagcatccacagcacagccaaggtggtgctgacccgcgaggacgttcactctcaagtcatctgcgaggtggcccacgtcaccttgcagggggaccctcttcgtgggactgccaacttgtctgagaccatccgagttccacccaccttggaggttactcaacagcccgtgagggcagagaaccaggtgaatgtcacctgccaggtgaggaagttctacccccagagactacagctgacctggttggagaatggaaacgtgtcccggacagaaacggcctcaacccttacagagaacaaggatggtacctacaactggatgagctggctcctggtgaatgtatctgcccacagggatgatgtgaagctcacctgccaggtggagcatgacgggcagccagcggtcagcaaaagccatgacctgaaggtctcagcccacccgaaggagcagggctcaaataccgccgctgagaacactggatctaatgaacggaacatctat

(19) United States
(12) Patent Application Publication
(10) Pub. No.: US 2011/0237498 A1
(43) Pub. Date: Sep. 29, 2011

(54) SOLUBLE POLYPEPTIDES FOR USE IN TREATING AUTOIMMUNE AND INFLAMMATORY DISORDERS

(75) Inventors: Marianne Raymond, La Prairie (CA); Marie Sarfati, Montreal (CA); Karl Welzenbach, Basel (CH); Maximilian Woisetschlaeger, Perchtoldsdorg (AT)

(73) Assignee: NOVARTIS AG, Basel (CH)

(21) Appl. No.: 13/132,400

(22) PCT Filed: Dec. 17, 2009
(86) PCT No.: PCT/EP2009/067411

§ 371 Date: Jun. 2, 2011

Related U.S. Application Data
(60) Provisional application No. 61/203,558, filed on Dec. 22, 2008.

(30) Foreign Application Priority Data
Dec. 19, 2008 (EP) 08172369.4
Dec. 23, 2008 (EP) 08172751.3

(51) Int. Cl. A61K 38/17; C07K 14/47; C07K 19/00; C07H 21/00; C12N 15/63; C12N 5/10; A61P 11/06; A61P 19/02; A61P 35/00; A61P 35/02; A61P 29/00; A61P 37/06; A61P 37/08; A61P 1/00; A61P 43/00; A61P 11/00

(52) U.S. Cl. 514/1.7; 530/350; 530/387.3; 514/21.2; 536/23.5; 435/320.1; 435/325; 435/358; 514/16.8; 514/19.3; 514/19.6

(57) ABSTRACT

The present invention relates to soluble CD47 binding polypeptides, for use as a medicament, in particular for the prevention or treatment of autoimmune and inflammatory disorders, for example allergic asthma and inflammatory bowel diseases. The invention more specifically relates to a soluble CD47 binding polypeptide for use as a medicament, comprising an extracellular domain of SIRPα (CD172a) or functional derivatives which bind to human CD47.

The present invention relates to soluble CD47 binding polypeptides, for use as a medicament, in particular for the prevention or treatment of autoimmune and inflammatory disorders, for example allergic asthma and inflammatory bowel diseases. The invention more specifically relates to a soluble CD47 binding polypeptide for use as a medicament, comprising an extracellular domain of SIRPα (CD172a) or functional derivatives which bind to human CD47.

CD47 is a cell surface glycoprotein which binds to SIRPα (alias SHPS-1) and SIRPγ on opposing cells. This interaction leads to negative regulation of immune cell function or can serve to mediating cellular adhesion and migration. CD47 was suggested for use as a biologics in the treatment of autoimmune disorders (WO1999/040940). In contrast, there are very few evidence of a potential use of CD47 ligands, such as SIRPα for similar therapeutic purposes. One explanation is the ubiquitous expression of CD47 that would prevent from using CD47 binding polypeptides as potential drugs. Data shown by Yu et al 2006 (J Invest Dermatol, 126, 797-807) suggest that a fusion protein made from the extracellular domains of SIRPα fused to an immunoglobulin Fc domain can prevent the migration from skin derived dendritic cells (DCs) to draining lymph nodes in mice and thereby attenuate (at least partially) contact hypersensitivity response in mice. Migration and function of DCs is essential for immune or inflammatory responses. Under disease condition these exacerbated responses of Des can lead to the perpetuation of disease. Interfering with migration of pathogenic DCs from tissue to lymphoid organs would be an attractive opportunity to stop the vicious cycle driving autoimmune- or inflammatory diseases. The present invention provides first in vivo evidence that SIRPα-Fc construct is suitable for preventing or stopping Th1/Th17- and Th2-driven diseases in animal models of disease. These data provide the basis for this invention and support the drugability of SIRPα-derived protein therapeutics. The invention is based, in part, on the discovery that the manipulation of CD47/SIRPα pathway suppresses immunogenic CD103⁻ dendritic cells-driven pathogenesis of Th1/Th17-(arthritis and colitis) as well as Th2 driven diseases (allergic asthma) These new findings offer previously unknown common mechanism on underlying causes of disease and represent therapeutic perspectives for multiple autoimmune and inflammatory disorders. In addition, recent evidence in published reports indicate that ligation of CD47 could be beneficial for treatment of several cancers (Majeti et al Cell 2009). While the reports indicate the use of CD47 antibodies, the invention here relates to the use of SIRPα-derived polypeptides for treatment of these diseases.

Therefore, in one aspect, the invention provides soluble CD47 binding polypeptides, for use as a medicament, comprising a SIRPα-derived polypeptide selected among the group consisting of a) an extracellular domain of SIR % (SEQ ID NO:3); b) a fragment of SEQ ID NO:1, and, c) a variant polypeptide of SEQ ID NO:1 having at least 75% identity to SEQ ID NO:3; wherein said SIRPα-derived polypeptide binds to human CD47 (SEQ ID NO:24). In certain embodiments, the variant polypeptide of SEQ ID NO:3 is at least 80%, 85%, 90%, 95% or 99% identical to SEQ ID NO:3.

For ease of reading, the soluble CD47 binding polypeptides according to the present invention are hereafter designated as the “Soluble Polypeptides of the Invention”.

In one embodiment, said SIRPα-derived polypeptide is selected among antagonist of CD47, i.e., a polypeptide that competitively inhibits the binding of a CD47 ligand to CD47, CD47 ligands include, without limitation, SIRPα, SIRPγ or TSP1.

In another embodiment, said SIRPα-derived polypeptide is selected among agonist of CD47, i.e., a polypeptide that is capable of inducing CD47 signaling activity

In one embodiment, said soluble CD47 binding polypeptide is selected among those that bind to human CD47 with a K_{D} of 2 μl or less and/or inhibits induced cytokine secretion as measured in an immune complex-stimulated dendritic cell cytokine release assay.

In another embodiment, said SIRPα-derived polypeptide is an extracellular domain of SIRPα comprising at least the V-region of SIRPα (SEQ ID NO:2).

In certain embodiments, the Soluble Polypeptide of the Invention is a fusion polypeptide comprising a first component consisting of a SIRPα-derived polypeptide fused to a second heterologous polypeptide. In one embodiment, the Soluble Polypeptide further comprises a spacer between the second heterologous polypeptide and the SIRPα-derived polypeptide. In one specific embodiment, the SIRPα derived polypeptide is fused to an IgG Fc domain. In a preferred embodiment, said Fc domain is a silent Fc fragment of human IgG1 isotype. In one embodiment, said Fc domain is an aglycosylated mutant variant of human IgG1 isotype.

In another related embodiment, the Soluble Polypeptides of the Invention are used as drugs in the treatment of autoimmune and inflammatory disorders. Preferred indications are selected among the group consisting of Th2-mediated airway inflammation, allergic disorders, asthma, inflammatory bowel diseases and ischemic disorders. In addition, the Soluble Polypeptides of the Invention may be used as drugs in the treatment of leukemias or cancer.

In order that the present invention may be more readily understood, certain terms are first defined. Additional definitions are set forth throughout the detailed description.

The term CD47 refers to human CD47. Human CD47 includes SEQ ID NO:24 but also any natural polymorphic, for example, comprising single nucleotide polymorphisms (SNPs), or splice variants of human CD47. Examples of splice variants or SNPs in CD47 nucleotide sequence found in human are described in Table 1.

TABLE 1
Variants of CD47 Protein

| Variant Type | Variant ID | Description |
|---|---|---|
| Splice Variant | NP_001768.1 | reference; longest variant; |
| | | sequence NO: 2 |
| | NP_942088.1 | different, shorter C-terminus |
| | NP_001020250.1 | different, shorter C-terminus |
| | ENSP00000381308 | different, shorter C-terminus |
| Single Nucleotide | rs11546646 | DNA: C or G; protein: A or P |
| Polymorphism | | (pos. 96 of NP_001768.1) |
| | ENSSNP12389584 | DNA: C or G; protein: V or L |
| | | (pos. 246 of NP_001768.1) |

The term SIRPα refers to the Signal Regulatory Protein Alpha (also designated CD172a or SHPS-1) which shows adhesion to CD47 integrin associated protein. In some embodiment, the term SIRPα refers to human SIRPα as defined in SEQ ID NO:23. Human SIRPα contains an amino acids extracellular domain (SEQ ID NO 3) with one V-type domain (SEQ ID NO 2) and two C1-type Ig domains and three potential N-glycosylation sites. It has a 110 amino acids cytoplasmic sequence with ITIM motifs that recruit tyrosine phosphatases SHP-1 and SHP-2 when phosphorylated. The term human SIRPα further includes, without limitation, any natural polymorphic, for example, comprising single nucleotide polymorphisms (SNPs), or splice variants of human SIRPα. Examples of splice variants or SNPs in SIR % nucleotide sequence found in human are described in Table 2.

TABLE 2
Variants of SIRPalpha Protein

| Variant Type | Variant ID | Description |
|---|---|---|
| Splice Variant | NP_542970.1 | reference; short variant; |
| | | sequence NO: 2 |
| | ENSP00000382941 | long variant, insertion of four |
| | | amino acids close to C-terminus |
| Single Nucleotide | rs17855609 | DNA: A or T; protein: T or S |
| Polymorphism | | (pos. 50 of NP_542970.1) |
| | rs17855610 | DNA: C or T; protein: T or I |
| | | (pos. 52 of NP_542970.1) |
| | rs17855611 | DNA: G or A; protein: R or H |
| | | (pos. 54 of NP_542970.1) |
| | rs17855612 | DNA: C or T; protein: A or V |
| | | (pos. 57 of NP_542970.1) |
| | rs1057114 | DNA: G or C; protein: G or A |
| | | (pos. 75 of NP_542970.1) |
| | rs1135200 | DNA: C or G; protein: D or E |
| | | (pos. 95 of NP_542970.1) |
| | rs17855613 | DNA: A or G; protein: N or D |
| | | (pos. 100 of NP_542970.1) |
| | rs17855614 | DNA: C or A; protein: N or K |
| | | (pos. 100 of NP_542970.1) |
| | rs17855615 | DNA: C or A; protein: R or S |
| | | (pos. 107 of NP_542970.1) |
| | rs1135202 | DNA: G or A; protein: G or S |
| | | (pos. 109 of NP_542970.1) |
| | rs17855616 | DNA: G or A; protein: G or S |
| | | (pos. 109 of NP_542970.1) |
| | rs2422666 | DNA: G or C; protein: V or L |
| | | (pos. 302 of NP_542970.1) |
| | rs12624995 | DNA: T or G; protein: V or G |
| | | (pos. 379 of NP_542970.1) |
| | rs41278990 | DNA: C or T; protein: P or S |
| | | (pos. 482 of NP_542970.1) |

As used herein, a polypeptide is “soluble” when it lacks any transmembrane domain or protein domain that anchors or integrates the polypeptide into the membrane of a cell expressing such polypeptide. In particular, the Soluble Polypeptides of the Invention may likewise exclude transmembrane and intracellular domains of SIRPα.

As used herein, a polypeptide that “binds to CD47” is intended to refer to a polypeptide that binds to human CD47 with a K_{D} of a 20 μM or less, 2 μM or less, 0.2M or less. In some embodiment, a polypeptide that binds to CD47 further binds to surfactant protein A (SP-A) and/or surfactant protein D (SP-D).

As used herein, a polypeptide that inhibits induced cytokine secretion as measured in a immune complex-stimulated dendritic cell cytokine release assay is a polypeptide that inhibits cytokine (e.g. IL-6, IL-10, IL-12p70, IL-23, IL-8 and/or TNF-α) release from peripheral blood monocytes, conventional dendritic cells (DCs) as well as monocyte-derived DCs stimulated with Staphylococcus Aureus Cowan 1 (Pansorbin) or soluble CD40L and IFN-γ. One example of an immune complex-stimulated dendritic cell cytokine release assay is described in more details in the examples below. In some embodiments, the Soluble Polypeptides of the invention inhibit cytokine secretion as measured in an immune complex-stimulated dendritic cell cytokine release assay at an IC₅₀ of 1 μM or less, 100 nM or less, or 10 nM or less.

As used herein, a polypeptide that inhibits T cell proliferation may be measured in a mixed lymphocyte reaction assay as described in the Example.

The term “K_{assoc}” or “Kₐ”, as used herein, is intended to refer to the association rate of a particular protein-protein interaction, whereas the term “K_{dis}” or “K_{d},” as used herein, is intended to refer to the dissociation rate of a particular protein-protein interaction. The term “K_{D}”, as used herein, is intended to refer to the dissociation constant, which is obtained from the ratio of K_{d} to Kₐ (i.e. K_{d}/Kₐ) and is expressed as a molar concentration (M). K_{D} values for protein-protein interaction can be determined using methods well established in the art. A method for determining the K_{D} of an protein/protein interaction is by using surface plasmon resonance, or using a biosensor system such as a Biacore® system.

As used herein, the term “Affinity” refers to the strength of interaction between the polypeptide and its target at a single site. Within each site the binding region of the polypeptide interacts through weak non-covalent forces with its target at numerous sites; the more interactions, the stronger the affinity.

As used herein, the term “high affinity” for a binding polypeptide refers to a polypeptide having a K_{D} of 10 nM or less, for example, 1 nM or less, for its target.

As used herein, the term “subject” includes any human or nonhuman animal.

The term “nonhuman animal” includes all vertebrates, e.g., mammals and non-mammals, such as nonhuman primates, sheep, dogs, cats, horses, cows, chickens, amphibians, reptiles, etc.

As used herein, the term, “optimized” means that a nucleotide sequence has been altered to encode an amino acid sequence using codons that are preferred in the production cell or organism, either a eukaryotic cell, for example, a cell of Pichia or Saccharomyces, a cell of Trichoderma, a Chinese Hamster Ovary cell (CHO) or a human cell, or a prokaryotic cell, for example, a strain of Escherichia coli.

The optimized nucleotide sequence is engineered to retain completely or as much as possible the amino acid sequence originally encoded by the starting nucleotide sequence, which is also known as the “parental” sequence. The optimized sequences herein have been engineered to have codons that are preferred in the corresponding production cell or organism, for example a mammalian cell, however optimized expression of these sequences in other prokaryotic or eukaryotic cells is also envisioned herein. The amino acid sequences encoded by optimized nucleotide sequences are also referred to as optimized.

Various aspects of the invention are described in further detail in the following subsections.

Assays to evaluate the effects of the Soluble Polypeptides of the Invention on functional properties of CD47 are described in further detail in the Examples.

### SIRPα-Derived Polypeptides

Soluble Polypeptides of the Invention comprise SIRPα-derived polypeptides that are selected among the group consisting of a) an extracellular domain of SIRPα (SEQ ID NO 3); b) a fragment of SEQ ID NO:3, and, c) a variant polypeptide of SEQ ID NO3; wherein said SIRPα-derived polypeptide binds to human CD47 (SEQ ID NO:24).

The Soluble Polypeptides of the Invention and their SIRPα-derived fragments should retain the capacity to bind to CD47. Fragments of SEQ ID NO:3 can therefore be selected among those fragments comprising the CD47 binding domain of SIRPα, Those fragments generally do not comprise the transmembrane and intracellular domains of SIRPα. In non-limiting illustrative embodiments, SIRPα-derived polypeptide essentially consists of SEQ ID NO:3 or SEQ ID NO:2, SIRPα-derived polypeptides further include, without limitation, variant polypeptides of SEQ ID NO:3 where amino acids residues have been mutated by amino acid deletion, insertion or substitution, yet have at least 60, 70, 80, 90 or 95 percent identity to SEQ ID NO:3; so long as changes to the native sequence do not substantially affect the biological activity of the molecule, in particular its binding to CD47. In some embodiments, it include mutant amino acid sequences wherein no more than 1, 2, 3, 4 or 5 amino acids have been mutated by amino acid deletion, insertion or substitution in the SIRPα-derived polypeptide when compared with SEQ ID NO:2. Examples of mutant amino acid sequences are those sequences derived from single nucleotide polymorphisms (see Table 2).

As used herein, the percent identity between the two sequences is a function of the number of identical positions shared by the sequences (i.e., % identity=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described below.

The percent identity between two amino acid sequences can be determined using the algorithm of E. Meyers and W. Miller (Comput. Appl. Biosci., 4:11-17, 1988) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol, 48:444-453, 1970) algorithm which has been incorporated into the GAP program in the GCG software package (available at http://www.gcg.com), using either a Blossom 62 matrix or a PAM250 matrix, and a gap weight of 16, 14, 12, 10, 8, 6, or 4 and a length weight of 1, 2, 3, 4, 5, or 6.

In a specific embodiment, the SIRPα-derived polypeptide includes changes to SEQ ID NO:13 or SEQ ID NO:2 that contain conservative amino acid substitutions.

Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, praline phenylalanine, methionine), beta-branched side chains (e.g., threonine; valine, isoleucine) and aromatic side chains (e.g., tyrosine, phenylalanine, tryptophan; histidine). Thus, one or more amino acid residues within the CD47 binding region of SIRPα-derived polypeptide can be replaced with other amino acid residues from the same side chain family, and the new polypeptide variant can be tested for retained function using the binding or functional assays described herein.

In some embodiments, the SIRPα-derived polypeptides are selected among those that retain the capacity to inhibit cytokine secretion as measured in a immune complex-stimulated dendritic cell cytokine release assay at least to the same extent as the polypeptide of SEQ ID NO:3 comprising the extracellular domain of human SIRPα.

In some embodiments, the SIRPα-derived polypeptides are selected among those that retain the capacity to inhibit T cell proliferation as measured in a mixed lymphocyte reaction assay.

In another embodiment, the SIRPα-derived polypeptides are selected among those that cross-react with non-human primate CD47.

### Fusion Polypeptides

In one aspect, the Soluble Polypeptides of the Invention are fusion polypeptides comprising the SIRPα-derived polypeptides.

In a preferred embodiment, the Soluble Polypeptides of the Invention are fusion polypeptides comprising the SIRPα-derived polypeptides and a second heterologous amino acid sequence, e.g, a portion of one or more proteins other than SIRPα, covalently bound to the SIRPα-derived polypeptide at the latter's N- and/or C-terminus, and optionally further comprising a linker.

The non SIRPα-derived protein can be preferably a soluble single chain polypeptide, which, when fused to another heterologous protein, is capable of increasing half life of the resulting fusion protein in blood. Alternatively or in addition, the non-SIRPα-derived protein comprises a domain for multimerization of the fusion polypeptide.

The non SIRPα-derived protein can be, for example, an immunoglobulin, serum albumin and fragments thereof. The non SIRPα-derived protein can also be a polypeptide capable of binding to serum albumin proteins to increase half life of the resulting molecule when administered in a subject. Such approach is for example described in Nygren et al., EP 0 486 525.

In one specific embodiment, the non-SIRPα derived protein is an Fc domain. The use of Fc moiety for making soluble construct with increased in vivo half life in human is well known in the art and for example described in Capon at al (U.S. Pat. No. 5,428,130).

As used herein the term “Fc domain” refers to the constant region of an immunoglobulin. An Fc domain comprises at least the CH2 and CH3 domain, optionally, the hinge region which is located between the heavy chain CH1 domain and CH2, Fc fragments could be obtained for example by papain digestion of an immunoglobulin. As used herein, the term Fc domain further include Fc variants into which a substitution, deletion or insertion of at least one amino acid has been introduced.

In one embodiment, the hinge region of CH1 is modified such that the number of cysteine residues in the hinge region is altered, e.g., increased or decreased. This approach is described further in U.S. Pat. No. 5,677,425 by Bodmer et al. The number of cysteine residues in the hinge region of CH1 is altered to, for example, facilitate assembly of the light and heavy chains or to increase or decrease the stability of the fusion polypeptide.

In another embodiment, the Fc region is modified to increase its biological half-life. Various approaches are possible. For example, one or more of the following mutations can be introduced; T252L, T254S, T256F, as described in U.S. Pat. No. 6,277,375 to Ward.

In yet other embodiments, the Fc region is altered by replacing at least one amino acid residue with a different amino acid residue to alter the effector functions of the Fc portion. For example, one or more amino acids can be replaced with a different amino acid residue such that the Fc portion has an altered affinity for an effector ligand. The effector ligand to which affinity is altered can be, for example, an Fc receptor or the C1 component of complement. This approach is described in further detail in U.S. Pat. Nos. 5,624,821 and 5,648,260, both by Winter at al.

In another embodiment, one or more amino acids selected from amino acid residues can be replaced with a different amino acid residue such that the resulting Fc portion has altered C1q binding and/or reduced or abolished complement dependent cytotoxicity (CDC). This approach is described in further detail in U.S. Pat. No. 6,194,551 by Idusogie et al.

In another embodiment, one or more amino acid residues are altered to thereby alter the ability of the Fc region to fix complement. This approach is described further in PCT Publication WO 94/29351 by Bodmer et al.

In yet another embodiment, the Fc region is modified to increase the ability of the fusion polypeptide to mediate antibody dependent cellular cytotoxicity (ADCC) and/or to increase or decrease the affinity of the Fc region for an Fey receptor by modifying one or more amino acids. This approach is described further in PCT Publication WO 00/42072 by Presta. Moreover, the binding sites on human IgG1 for FcγRI, FcγRII, FcγRIII and FcRn have been mapped and variants with improved binding have been described (see Shields, R. L. et al., 2001 J. Biol. Chem. 276:6591-6604).

In one embodiment, the Fc domain is of human origin and may be from any of the immunoglobulin classes, such as IgG or IgA and from any subtype such as human IgG1, IgG2, IgG3 and IgG4. In other embodiments the Fc domain is from a non-human animal, for example, but not limited to a mouse, rat, rabbit, camel, shark, non-human primate or hamster.

In certain embodiments, the Fc domain of IgG1 isotype is used. In some specific embodiments, a mutant variant of IgG1 Fc fragment is used, e.g. a silent IgG1 Fc which reduces or eliminates the ability of the fusion polypeptide to mediate antibody dependent cellular cytotoxicity (ADCC) and/or to bind to an Fey receptor. An example of an IgG1 isotype silent mutant, is a so-called LALA mutant, wherein Leucine residue is replaced by Alanine residue at amino acid positions 234 and 235 as described in J. Virol 2001 December; 75(24):12161-8 by Hezareh et al. In certain embodiments, the Fc domain is a mutant preventing glycosylation at residue at position 297 of Fc domain. For example, an amino acid substitution of asparagine residue at position 297 of the Fc domain. Example of such amino acid substitution is the replacement of N297 by a glycine or an alanine.

In one embodiment, the Fc domain comprises a dimerization domain, preferably via cysteine capable of making covalent disulfide bridge between two fusion polypeptides comprising such Fc domain.

The SIRPα-derived polypeptide can be fused directly in frame with the non-SIRPα-derived protein or via a polypeptidic linker (spacer). Such spacer may be a single amino acid (such as for example, a glycine residue) or between 5-100 amino acids, for example between 5-20 amino acids. The linker should permit the SIRPα-derived domain to assume the proper spatial orientation to form a binding site with CD47. Suitable polypeptide linkers may be selected among those that adopt a flexible conformation. Examples of such linkers are (without limitation) those linkers comprising Glycine and Serine residues, for example, (Gly₄Ser)ₙ, wherein n=1-12.

### Glycosylation Modifications

In still another embodiment, the glycosylation pattern of the Soluble Polypeptide of the Invention can be altered compared to typical mammalian glycosylation pattern such as those obtained in CHO or human cell lines. For example, an aglycoslated polypeptides can be made by using prokaryotic cell lines as host cells or mammalian cells that has been engineered to lack glycosylation. Carbohydrate modifications can also be accomplished by for example, altering one or more sites of glycosylation within the Soluble Polypeptide.

Additionally or alternatively, a glycosylated polypeptide can be made that has an altered type of glycosylation. Such carbohydrate modifications can be accomplished by, for example, expressing the Soluble Polypeptides in a host cell with altered glycosylation machinery, i.e the glycosylation pattern of the Soluble Polypeptide is altered compared to the glycosylation pattern observed in corresponding wild type cells. Cells with altered glycosylation machinery have been described in the art and can be used as host cells in which to express recombinant Soluble Polypeptides of the Invention to thereby produce such Soluble Polypeptides with altered glycosylation For example, EP 1,176,195 by Hang at al., describes a cell line with a functionally disrupted FUT8 gene, which encodes a fucosyl transferase, such that glycoproteins expressed in such a cell line exhibit hypofucosylation. PCT Publication WO 03/035835 by Presta describes a variant CHO cell line, LecI3 cells, with reduced ability to attach fucose to Asn(297)-linked carbohydrates, also resulting in hypofucosylation of glycoproteins expressed in that host cell (see also Shields, R. L., et al., 2002 J. Biol. Chem. 277:26733-26740). Alternatively, the Soluble Polypeptides of the Invention can be produced in yeast, e.g., Pichia pastoris, or filamentous fungi, e.g., Trichoderma reesei engineered for mammalian-like glycosylation pattern (see for example EP129717281). Advantages of those glycoengineered host cells are, inter alia, the provision of polypeptide compositions with homogeneous glycosylation pattern and/or higher yield.

### Pegylated Soluble Polypeptides and Other Conjugates

Another embodiment of the Soluble Polypeptides herein that is contemplated by the invention is pegylation. The Soluble Polypeptides of the invention, for example, consisting essentially of SIRPα-derived polypeptides can be pegylated. Pegylation is a well-known technology to increase the biological (e.g., serum) half-life of the resulting biologics as compared to the same biologics without pegylation. To pegylate a polypeptide, the polypeptide is typically reacted with polyethylene glycol (PEG), such as a reactive ester or aldehyde derivative of PEG, under conditions in which one or more PEG groups become attached to the polypeptides. The pegylation can be carried out by an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide. Methods for pegylating proteins are known in the art and can be applied to the Soluble Polypeptides of the invention. See for example, EP 0 154 316 by Nishimura at al. and EP 0 401 384 by Ishikawa et al.

Alternative conjugates or polymeric carrier can be used, in particular to improve the pharmacokinetic properties of the resulting conjugates. The polymeric carrier may comprise at least one natural or synthetic branched, linear or dendritic polymer. The polymeric carrier is preferably soluble in water and body fluids and is preferably a pharmaceutically acceptable polymer. Water soluble polymer moieties include, but are not limited to e.g. polyalkylene glycol and derivatives thereof, including PEG, PEG homopolymers, mPEG, polypropyleneglycol homopolymers, copolymers of ethylene glycol with propylene glycol, wherein said homopolymers and copoloymers are unsubstituted or substituted at one end e.g. with an acylgroup; polyglycerines or polysialic acid: carbohydrates polysaccharides, cellulose and cellulose derivatives, including methylcellulose and carboxymethylcellulose; starches (e.g. hydroxyalkyl starch (HAS), especially hydroxyethyl starch (HES) and dextrines, and derivatives thereof: dextran and dextran derivatives, including dextransulfat, crosslinked dextrin, and carboxymethyl dextrin: chitosan (a linear polysaccharide), heparin and fragments of heparin; polyvinyl alcohol and polyvinyl ethyl ethers; polyvinylpyrrollidon; alpha, beta-poly[(2-hydroxyethyl)-DL-aspartamide; and polyoxy-ethylated polyols.

### Use of the Soluble Polypeptides as a Medicament

The Soluble Polypeptides of the Invention have been shown to protect from inflammatory disorders such as allergic asthma or inflammatory bowel diseases in animal model and therefore may be used as a medicament, in particular to decrease or suppress (in a statistically or biologically significant manner) the inflammatory and/or autoimmune response, in particular, a response mediated by SIRPα+ cells in a subject.

Nucleic acid molecules encoding the Soluble Polypeptides of the Invention

Another aspect of the invention pertains to nucleic acid molecules that encode the Soluble Polypeptides of the invention or at least SIRPα-derived polypeptides, including without limitation, the embodiments related to fusion polypeptides. Examples of nucleotide sequences encoding Soluble Polypeptides of the Invention comprise SEQ ID NOs: 26 or 27.

The nucleic acids may be present in whole cells, in a cell lysate, or may be nucleic acids in a partially purified or substantially pure form. A nucleic acid is “isolated” or “rendered substantially pure” when purified away from other cellular components or other contaminants, e.g., other cellular nucleic acids or proteins, by standard techniques, including alkaline/SDS treatment, CsCl banding, column chromatography, agarose gel electrophoresis and others well known in the art. See, F. Ausubel, et al., ed. 1987 Current Protocols in Molecular Biology, Greene Publishing and Wiley Interscience, New York. A nucleic acid of the invention can be, for example, DNA or RNA and may or may not contain intronic sequences. In an embodiment, the nucleic acid is a cDNA molecule. The nucleic acid may be present in a vector such as a phage display vector, or in a recombinant plasmid vector.

Once DNA fragments encoding the Soluble Polypeptides of the Invention are obtained, for example, fusion polypeptide comprising the SIRPα-derived polypeptides as described above, these DNA fragments can be further manipulated by standard recombinant DNA techniques, for example to include any signal sequence for appropriate secretion in expression system, any purification tag and cleavable tag for further purification steps. In these manipulations, a DNA fragment is operatively linked to another DNA molecule, or to a fragment encoding another protein, such as a purification/secretion tag or a flexible linker. The term “operatively linked”, as used in this context, is intended to mean that the two DNA fragments are joined in a functional manner, for example, such that the amino acid sequences encoded by the two DNA fragments remain in-frame, or such that the protein is expressed under control of a desired promoter.

### Generation of Transfectomas Producing the SIRPα-Derived Polypeptide or Soluble Polypeptides

The Soluble Polypeptides of the Invention can be produced in a host cell transfectoma using, for example, a combination of recombinant DNA techniques and gene transfection methods as is well known in the art.

For example, to express the Soluble Polypeptides of Invention or intermediates thereof, such as SIRPα-derived polypeptides, DNAs encoding the corresponding polypeptides, can be obtained by standard molecular biology techniques (e.g., PCR amplification or cDNA cloning using a hybridoma that expresses the polypeptides of interest) and the DNAs can be inserted into expression vectors such that the corresponding gene is operatively linked to transcriptional and translational control sequences. In this context, the term “operatively linked” is intended to mean that a gene is ligated into a vector such that transcriptional and translational control sequences within the vector serve their intended function of regulating the transcription and translation of the gene. The expression vector and expression control sequences are chosen to be compatible with the expression host cell used. The gene encoding Soluble Polypeptides or intermediates are inserted into the expression vector by standard methods (e.g., ligation of complementary restriction sites on the gene fragment and vector, or blunt end ligation if no restriction sites are present). Additionally or alternatively, the recombinant expression vector can encode a signal peptide that facilitates secretion of the polypeptide chain from a host cell. The gene can be cloned into the vector such that the signal peptide is linked in frame to the amino terminus of the polypeptide chain. The signal peptide can be a SIRPα signal peptide or a heterologous signal peptide (i.e., a signal peptide not naturally associated with SIRPα sequence).

In addition to the polypeptide encoding sequence, the recombinant expression vectors of the invention carry regulatory sequences that control the expression of the gene in a host cell. The term “regulatory sequence” is intended to include promoters, enhancers and other expression control elements (e.g., polyadenylation signals) that control the transcription or translation of the polypeptide chain genes. Such regulatory sequences are described, for example, in Goeddel (Gene Expression Technology. Methods in Enzymology 185, Academic Press, San Diego, Calif. 1990). It will be appreciated by those skilled in the art that the design of the expression vector, including the selection of regulatory sequences, may depend on such factors as the choice of the host cell to be transformed, the level of expression of protein desired, etc. Regulatory sequences for mammalian host cell expression include viral elements that direct high levels of protein expression in mammalian cells, such as promoters and/or enhancers derived from cytomegalovirus (CMV), Simian Virus 40 (SV40), adenovirus (e.g., the adenovirus major late promoter (AdMLP)), and polyoma. Alternatively, nonviral regulatory sequences may be used such as the ubiquitin promoter or P-globin promoter. Still further, regulatory elements composed of sequences from different sources, such as the SRa promoter system, which contains sequences from the SV40 early promoter and the long terminal repeat of human T cell leukemia virus type (Takebe, Y. et al., 1988 Mol. Cell. Biol. 8:466-472).

In addition to this, the recombinant expression vectors of the invention may carry additional sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216, 4,634,665 and 5,179,017, all by Axel et al.). For example, typically the selectable marker gene confers resistance to drugs, such as G418, hygromycin or methotrexate, on a host cell into which the vector has been introduced. Selectable marker genes include the dihydrofolate reductase (DHFR) gene (for use in dhfr− host cells with methotrexate selection/amplification) and the neo gene (for G418 selection).

For expression of the polypeptide, the expression vector(s) encoding the Soluble Polypeptide or intermediates such as SIRPα-derived polypeptide is transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. It is theoretically possible to express the Soluble Polypeptides of the Invention in either prokaryotic or eukaryotic host cells. Expression of glycoprotein in eukaryotic cells, in particular mammalian host cells, is discussed because such eukaryotic cells, and in particular mammalian cells, are more likely than prokaryotic cells to assemble and secrete a properly folded and biologically active glycoprotein such as the Soluble Polypeptides of the Invention.

Mammalian host cells for expressing the Soluble Polypeptides and intermediates such as SIRPα-derived polypeptides of the invention include Chinese Hamster Ovary (CHO cells) (including dhfr− CHO cells, described Urlaub and Chasin, 1980 Proc. Natl. Acad. Sci. USA 77:4216-4220 used with a DH FR selectable marker, e.g., as described in R. J. Kaufman and P. A. Sharp. 1982 Mol. Biol. 159:601-621, NSO myeloma cells, COS cells and SP2 cells) or human cell lines (including PER-C6 cell lines, Crucell). In particular, for use with NSO myeloma cells, another expression system is the GS gene expression system shown in WO 87/04462, WO 89/01036 and EP 338,841. When recombinant expression vectors encoding polypeptides are introduced into mammalian host cells, the Soluble Polypeptides or intermediates such as SIRPα-derived polypeptides are produced by culturing the host cells for a period of time sufficient to allow for expression of the recombinant polypeptides in the host cells or secretion of the recombinant polypeptides into the culture medium in which the host cells are grown. The polypeptides can then be recovered from the culture medium using standard protein purification methods.

### Multivalent Proteins

in another aspect, the present invention provides multivalent proteins comprising at least two identical or different Soluble Polypeptides of the Invention binding to CD47. In one embodiment, the multivalent proteins comprise at least two three or four Soluble Polypeptides of the Invention. The Soluble CD47-binding Polypeptides can be linked together via protein fusion or covalent or non covalent linkage. The multivalent proteins of the present invention can be prepared by conjugating the constituent binding specificities, using methods known in the art. For example, each binding specificity of the multivalent protein can be generated separately and then conjugated to one another.

A variety of coupling or cross-linking agents can be used for covalent conjugation. Examples of cross-linking agents include protein A, carbodiimide, N-succinimidyl-S-acetyl-thioacetate (SATA). 5,5′-dithiobis(2-nitrobenzoic acid) (DTNB), o-phenylenedimaleimide (oPDM), N-succinimidyl-3-(2-pyridyldithio)propionate (SPDP), and sulfosuccinimidyl 4-(N-maleimidomethyl)cyclohexane-I-carboxylate (sulfo-SMCC) (see e.g., Karpoveky et al., 1984 J. Exp. Med. 160:1686; Liu, M A et al., 1985 Proc. Natl. Acad. Sci. USA 82:8648). Other methods include those described in Paulus, 1985 Behring Ins. Mitt. No. 78, 118-132: Brennan et al., 1985 Science 229:81-83), and Glennie et al., 1987 J. Immunol. 139: 2367-2375). Covalent linkage can be obtained by disulfide bridge between two cysteines, for example disulfide bridge from cysteine of an Fc domain.

In a particular embodiment, the hinge region of an Fc domain fused to SIRPα-derived polypeptide is modified to contain an odd number of sulfhydryl residues, for example one, prior to conjugation. Alternatively, both binding specificities can be encoded in the same vector and expressed and assembled in the same host cell.

### Pharmaceutical Compositions

in another aspect, the present invention provides a composition, e.g., a pharmaceutical composition, containing one or a combination of the Soluble Polypeptides of the present Invention, formulated together with a pharmaceutically acceptable carrier.

pharmaceutical formulations comprising a soluble polypeptide of the invention may be prepared for storage by mixing the polypeptide having the desired degree of purity with optional physiologically acceptable carriers, excipients or stabilizers {Remington: The Science and Practice of Pharmacy 20th edition (2000)) in the form of aqueous solutions, lyophilized or other dried formulations. Therefore, the invention further relates to a lyophilized composition comprising at least the Soluble Polypeptide of the invention.

Pharmaceutical compositions of the invention also can be administered in combination therapy, i.e., combined with other agents. For example, the combination therapy can include a Soluble Polypeptide of the present Invention combined with at least one other anti-inflammatory or another chemotherapeutic agent. Examples of therapeutic agents that can be used in combination therapy are described in greater detail below in the section on uses of the Soluble Polypeptides of the Invention.

As used herein, “pharmaceutically acceptable carrier” includes any and all solvents, dispersion media, coatings, antibacterial and antifungal agents, isotonic and absorption delaying agents, and the like that are physiologically compatible. The carrier should be suitable for intravenous, intramuscular, subcutaneous, parenteral, spinal or epidermal administration (e.g., by injection or infusion). Depending on the route of administration, the active compound may be coated in a material to protect the compound from the action of acids and other natural conditions that may inactivate the compound.

The pharmaceutical compounds of the invention may include one or more pharmaceutically acceptable salts. A “pharmaceutically acceptable salt” refers to a salt that retains the desired biological activity of the parent compound and does not impart any undesired toxicological effects (see e.g., Berge, S. M., et al., 1977 J. Pharm. Sci. 6611-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and di-carboxylic acids phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

A pharmaceutical composition of the invention also may include a pharmaceutically acceptable anti-oxidant. Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, one can include isotonic agents, for example, sugars, polyalcohols such as mannitol, sorbitol, or sodium chloride in the composition. Prolonged absorption of the injectable compositions can be brought about by including in the composition an agent that delays absorption for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the methods of preparation are vacuum drying and freeze-drying (lyophilization) that yield a powder of the active ingredient plus any additional desired ingredient from a previously sterile-filtered solution thereof.

The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. Generally, out of one hundred percent, this amount will range from about 0.01 percent to about ninety-nine percent of active ingredient, from about 0.1 percent to about 70 percent, or from about 1 percent to about 30 percent of active ingredient in combination with a pharmaceutically acceptable carrier.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of sensitivity in individuals.

For administration of the Soluble Polypeptides of the Invention, the dosage ranges from about 0.0001 to 100 mg/kg, and more usually 0.01 to 5 mg/kg, of the host body weight. For example dosages can be 0.3 mg/kg body weight, 1 mg/kg body weight, 3 mg/kg body weight, 5 mg/kg body weight or 10 mg/kg body weight or within the range of 1-10 mg/kg. An exemplary treatment regime entails administration once per week, once every two weeks, once every three weeks, once every four weeks, once a month, once every 3 months or once every three to 6 months. Dosage regimens for a Soluble Polypeptide of the Invention include 1 mg/kg body weight or 3 mg/kg body weight by intravenous administration, with the polypeptide being given using one of the following dosing schedules: every four weeks for six dosages, then every three months; every three weeks; 3 mg/kg body weight once followed by 1 mg/kg body weight every three weeks.

The Soluble Polypeptide is usually administered on multiple occasions. Intervals between single dosages can be, for example, weekly, monthly, every three months or yearly. Intervals can also be irregular as indicated by measuring blood levels of Soluble Polypeptide in the patient. In some methods, dosage is adjusted to achieve a plasma polypeptide concentration of about 1-1000 μg/ml and in some methods about 25-300 μg/ml.

Alternatively, the Soluble Polypeptide can be administered as a sustained release formulation, in which case less frequent administration is required. Dosage and frequency vary depending on the half-life of the Soluble Polypeptide in the patient. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated or until the patient shows partial or complete amelioration of symptoms of disease. Thereafter, the patient can be administered a prophylactic regime.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A “therapeutically effective dosage” of Soluble Polypeptide of the Invention can result in a decrease in severity of disease symptoms, an increase in frequency and duration of disease symptom-free periods, or a prevention of impairment or disability due to the disease affliction.

A composition of the present invention can be administered by one or more routes of administration using one or more of a variety of methods known in the art. As will be appreciated by the skilled artisan, the route and/or mode of administration will vary depending upon the desired results. Routes of administration for Soluble Polypeptides of the Invention include intravenous, intramuscular, intradermal, intraperitoneal, subcutaneous, spinal or other parenteral routes of administration, for example by injection or infusion. The phrase “parenteral administration” as used herein means modes of administration other than enteral and topical administration usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Alternatively, a Soluble Polypeptide of the Invention can be administered by a nonparenteral route, such as a topical, epidermal or mucosal route of administration, for example, intranasally, orally, vaginally, rectally, sublingually or topically.

The active compounds can be prepared with carriers that will protect the compound against rapid release, such as a controlled release formulation, including implants, transdermal patches, and microencapsulated delivery systems. Biodegradable biocompatible polymers can be used such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc.; New York, 1978.

Therapeutic compositions can be administered with medical devices known in the art. For example, in one embodiment, a therapeutic composition of the invention can be administered with a needleless hypodermic injection device, such as the devices shown in U.S. Pat. Nos. 5,399,163; 5,383,851; 5,312,335; 5,064,413; 4,941,880; 4,790,824 or 4,596,556. Examples of well known implants and modules useful in the present invention include. U.S. Pat. No. 4,487,603, which shows an implantable micro-infusion pump for dispensing medication at a controlled rate; U.S. Pat. No. 4,486,194, which shows a therapeutic device for administering medicants through the skin; U.S. Pat. No. 4,447,233, which shows a medication infusion pump for delivering medication at a precise infusion rate: U.S. Pat. No. 4,447,224, which shows a variable flow implantable infusion apparatus for continuous drug delivery; U.S. Pat. No. 4,439,196, which shows an osmotic drug delivery system having multi-chamber compartments and U.S. Pat. No. 4,475,196, which shows an osmotic drug delivery system. Many other such implants, delivery systems, and modules are known to those skilled in the art.

In certain embodiments, the Soluble Polypeptides of the Invention can be formulated to ensure proper distribution in vivo. For example, the blood-brain barrier (BBB) excludes many highly hydrophilic compounds. To ensure that the therapeutic compounds of the invention cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs, thus enhance targeted drug delivery (see, e.g., V. V, Ranade, 1989 J. Cline Pharmacol, 29685). Exemplary targeting moieties include foists or biotin (see, e.g., U.S. Pat. No. 5,416,016 to Low et al.): mannosides (Umezawa et al., 1988 Biochem. Biophys. Res. Commun. 153:1038) antibodies (P. G. Bloeman et al., 1995 FEBS Lett. 357:140: M. Owais et al., 1995 Antimicrob. Agents Chemother 391180); surfactant protein A receptor (Briscoe et al., 1995 Am. J. Physiol. 1233:134); p120 (Schreier et al., 1994 J. Biol. Chem., 269:9090) see also K. Keinanen; M. L. Laukkanen, 1994 FEBS Lett. 346:123; J. J. Killion; I. J. Fidler, 1994 Immunomethods 4:273.

### Uses and Methods of the Invention

The Soluble Polypeptides of the Invention have in vitro and in vivo diagnostic and therapeutic utilities. For example, these molecules can be administered to cells in culture, e.g. in vitro or in vivo, or in a subject, e.g., in vivo, to treat, prevent or diagnose a variety of disorders.

The term “subject” as used herein is intended to include human and non-human animals. Non-human animals include all vertebrates, e.g., mammals and non-mammals, such as non-human primates, sheep, dogs, cats, cows, horses, chickens, amphibians, and reptiles.

The methods are particularly suitable for treating, preventing or diagnosing autoimmune and inflammatory disorders mediated by SIRPα+ cells, e.g., allergic asthma or ulcerative colitis. These include inflammatory conditions, allergies and allergic conditions, autoimmune diseases, ischemic disorders, severe infections, and cells, organ or tissue transplant rejection, including xenotransplant (i.e. transplant from different species, for example from non-human species to human) of cells, tissues or organs.

Examples of autoimmune diseases includes, without limitation, arthritis (for example rheumatoid arthritis, arthritis chronica progrediente and arthritis deformans) and rheumatic diseases, including inflammatory conditions and rheumatic diseases involving bone loss, inflammatory pain, spondyloarhropathies including ankylosing spondylitis, Reiter syndrome, reactive arthritis, psoriatic arthritis, and enterophathis arthritis, hypersensitivity (including both airways hypersensitivity and dermal hypersensitivity) and allergies. Autoimmune diseases include autoimmune haematological disorders (including e.g. hemolytic anaemia, aplastic anaemia, pure red cell anaemia and idiopathic thrombocytopenia), systemic lupus erythematosus, inflammatory muscle disorders, polychondritis, sclerodoma, Wegener granulomatosis dermatomyositis, chronic active hepatitis, myasthenia gravis, psoriasis, Steven-Johnson syndrome, idiopathic sprue, endocrine ophthalmopathy, Graves disease, sarcoidosis, multiple sclerosis, primary biliary cirrhosis, juvenile diabetes (diabetes mellitus type I), uveitis (anterior and posterior), keratoconjunctivitis sicca and vernal keratoconjunctivitis, interstitial lung fibrosis, psoriatic arthritis and glomerulonephritis (with and without nephrotic syndrome, e.g. including idiopathic nephrotic syndrome or minimal change nephropathy), tumors, multiple sclerosis, inflammatory disease of skin and cornea, myositis, loosening of bone implants, metabolic disorders, such as atherosclerosis, diabetes, and dislipidemia.

The Soluble Polypeptides of the Invention are also useful for the treatment, prevention, or amelioration of asthma, bronchitis, pneumoconiosis, pulmonary emphysema, and other obstructive or inflammatory diseases of the airways.

The Soluble Polypeptides of the Invention are also useful for the treatment of IgE-mediated disorders. IgE mediated disorders include atopic disorders, which are characterized by an inherited propensity to respond immunologically to many common naturally occurring inhaled and ingested antigens and the continual production of IgE antibodies. Specific atopic disorders includes allergic asthma, allergic rhinitis, atopic dermatitis and allergic gastroenteropathy.

However, disorders associated with elevated IgE levels are not limited to those with an inherited (atopic) etiology. Other disorders associated with elevated IgE levels, that appear to be IgE-mediated and are treatable with the formulations of this present invention include hypersensitivity (e.g., anaphylactic hypersensitivity), eczema, urticaria, allergic bronchopulmonary aspergillosis, parasitic diseases, hyper-IgE syndrome, ataxia-telangiectasia, Wiskott-Aldrich syndrome, thymic alymphoplasia, IgE myeloma and graft-versus-host reaction.

The Soluble Polypeptides of the Invention may be administered as the sole active ingredient or in conjunction with, e.g. as an adjuvant to or in combination to, other drugs e.g. immunosuppressive or immunomodulating agents or other anti-inflammatory agents, e.g. for the treatment or prevention of diseases mentioned above. For example, the Soluble Polypeptides of the invention may be used in combination with DMARD, e.g. Gold salts, sulphasalazine, antimalarias, methotrexate, D-penicillamine, azathioprine, mycophenolic acid, cyclosporine A, tacrolimus, sirolimus minocycline, leflunomide, glucocorticoids: a calcineurin inhibitor, e.g. cyclosporin A or FK 506 a modulator of lymphocyte recirculation, e.g. FTY720 and FTY720 analogs: a mTOR inhibitor, e.g. rapamycin, 40-O-(2-hydroxyethyl)-rapamycin CCI779, ABT578, AP23573 or TAFA-93′, an ascomycin having immuno-suppressive properties, e.g. ABT-281, ASM981, etc., corticosteroids, oyclo-phos-phamide; azathioprene; methotrexate; leflunomide; mizoribine; mycophenolic acid; myco-pheno-late mofetil, 15-deoxyspergualine or an immunosuppressive homologue, analogue or derivative thereof; immunosuppressive monoclonal antibodies, e.g., monoclonal antibodies to leukocyte receptors, e.g., MHC, CD2, CD3, CD4, CD7, CD8, CD25, CD28, CD40. CD45, CD58, CD80, CD86 or their ligands: other immunomodulatory compounds, e.g. a recombinant binding molecule having at least a portion of the extracellular domain of CTLA4 or a mutant thereof, e.g. an at least extracellular portion of CTLA4 or a mutant thereof joined to a non-CTLA4 protein sequence, e.g. CTLA4Ig (for ex. designated ATCC 68629) or a mutant thereof, e.g. LEA29Y; adhesion molecule inhibitors, e.g. LFA-1 antagonists, ICAM-1 or -3 antagonists, VCAM-4 antagonists or VLA-4 antagonists; or a chemotherapeutic agent, e.g. paclitaxel, gemcitabine, cisplatinum, doxorubicin or 5-fluorouracil; anti TNF agents, e.g. monoclonal antibodies to TNF, e.g. infliximab, adalimumab, CDP870, or receptor constructs to TNF-RI or TNF-RII, e.g. Etanercept, PEG-TNF-RI; blockers of proinflammatory cytokines, IL-1 blockers, e.g. Anakinra or IL-1 trap, AAL160, ACZ 885, IL-6 blockers; chemokines blockers, e.g inhibitors or activators of proteases, e.g. metalloproteases, anti-IL-15 antibodies, anti-IL-6 antibodies, anti-CD20 antibodies, anti-CD22 antibodies, anti-IL17 antibodies, NSAIDs, such as aspirin or an anti-infectious agent (list not limited to the agent mentioned).

The Soluble Polypeptides of the Invention are also useful as co-therapeutic agents for use in conjunction with anti-inflammatory or bronchodilatory drug substances, particularly in the treatment of obstructive or inflammatory airways diseases such as those mentioned hereinbefore, for example as potentiators of therapeutic activity of such drugs or as a means of reducing required dosaging or potential side effects of such drugs. An agent of the invention may be mixed with the anti-inflammatory or bronchodilatory drug in a fixed pharmaceutical composition or it may be administered separately, before, simultaneously with or after the anti-inflammatory or bronchodilatory drug. Such anti-inflammatory drugs include steroids, in particular glucocorticosteroids such as budesonide, beclamethasone, fluticasone or mometasone, and dopamine receptor agonists such as cabergoline, bromocriptine or ropinirole. Such bronchodilatory drugs include anticholinergic or antimuscarinic agents, in particular ipratropium bromide, oxitropium bromide and tiotropium bromide.

Combinations of agents of the invention and steroids may be used, for example, in the treatment of COPD or particularly, asthma. Combinations of agents of the invention and anticholinergic or antimuscarinic agents or dopamine receptor agonists may be used, for example, in the treatment of asthma or, particularly, COPD.

In accordance with the foregoing, the present invention also provides a method for the treatment of an obstructive or inflammatory airways disease which comprises administering to a subject, particularly a human subject, in need thereof a Soluble Polypeptide, as hereinbefore described. In another aspect, the invention provides a Soluble Polypeptide, as hereinbefore described for use in the preparation of a medicament for the treatment of an obstructive or inflammatory airways disease.

The Soluble Polypeptides of the Invention are also particularly useful for the treatment, prevention, or amelioration of chronic gastrointestinal inflammation, such as inflammatory bowel diseases, including Crohn's disease and ulcerative colitis.

“Chronic gastrointestinal inflammation” refers to inflammation of the mucosal of the gastrointestinal tract that is characterized by a relatively longer period of onset, is long-lasting (e.g., from several days, weeks, months, or years and up to the life of the subject), and is associated with infiltration or influx of mononuclear cells and can be further associated with periods of spontaneous remission and spontaneous occurrence. Thus, subjects with chronic gastrointestinal inflammation may be expected to require a long period of supervision, observation, or care, “Chronic gastrointestinal inflammatory conditions” (also referred to as “chronic gastrointestinal inflammatory diseases”) having such chronic inflammation include, but are not necessarily limited to inflammatory bowel disease (1BD), colitis induced by environmental insults (e.g., gastrointestinal inflammation (e.g., colitis) caused by or associated with (e.g., as a side effect) a therapeutic regimen, such as administration of chemotherapy, radiation therapy, and the like), colitis in conditions such as chronic granulomatous disease (Schappi et al. Arch Dis Child, 2001 February; 1984(2):147-151), celiac disease, celiac sprue (a heritable disease in which the intestinal lining is inflamed in response to the ingestion of a protein known as gluten), food allergies, gastritis, infectious gastritis or enterocolitis (e.g., Helicobacter pylori-infected chronic active gastritis) and other forms of gastrointestinal inflammation caused by an infectious agent, and other like conditions.

As used herein, “inflammatory bowel disease” or “IBD” refers to any of a variety of diseases characterized by inflammation of all or part of the intestines. Examples of inflammatory bowel disease include, but are not limited to Crohn's disease and ulcerative colitis, Reference to IBD throughout the specification is often referred to in the specification as exemplary of gastrointestinal inflammatory conditions, and is not meant to be limiting.

In accordance with the foregoing, the present invention also provides a method for the treatment of chronic gastrointestinal inflammation or inflammatory bowel diseases, such as ulcerative colitis, which comprises administering to a subject, particularly a human subject, in need thereof, a Soluble Polypeptide, as hereinbefore described. In another aspect, the invention provides a Soluble Polypeptide, as hereinbefore described for use in the preparation of a medicament for the treatment of chronic gastrointestinal inflammation or inflammatory bowel diseases.

The present invention is also useful in the treatment, prevention or melioration of leukemias or other cancer disorders.

Also encompassed within the scope of the present invention is a method as defined above comprising co-administration, e.g. concomitantly or in sequence, of a therapeutically effective amount of a Soluble Polypeptide, and at least one second drug substance, said second drug substance being a immuno-suppressive immunomodulatory, anti-inflammatory chemotherapeutic or anti-infectious drug, e.g. as indicated above.

Or, a therapeutic combination, e.g. a kit, comprising of a therapeutically effective amount of a) a Soluble Polypeptide of the Invention and b) at least one second substance selected from a immuno-suppressive immunomodulatory, anti-inflammatory chemotherapeutic or anti-infectious drug, e.g. as indicated above. The kit may comprise instructions for its administration.

Where the Soluble Polypeptides of the Invention are administered in conjunction with other immuno-suppressive/immunomodulatory, anti-inflammatory chemotherapeutic or anti-infectious therapy, dosages of the co-administered combination compound will of course vary depending on the type of co-drug employed, on the condition being treated and so forth.

In one embodiment, the Soluble Polypeptides of the invention can be used to detect levels of CD47+ dendritic cells, or levels of cells that contain CD47. This can be achieved, for example, by contacting a sample (such as an in vitro sample) and a control sample with a Soluble Polypeptide of the Invention under conditions that allow for the formation of a complex between the Soluble Polypeptides and CD47 expressing cells. Any complexes formed are detected and compared in the sample and the control. For example, standard detection methods, well known in the art, such as flow cytometric assays, can be performed using the compositions of the invention.

Accordingly, in one aspect, the invention further provides methods for detecting the presence of CD47 (e.g., human CD47) in a sample, or measuring the amount of CD47, comprising contacting the sample, and a control sample, with a Soluble Polypeptide of the Invention, under conditions that allow for formation of a complex between the Soluble Polypeptide and CD47. The formation of a complex is then detected, wherein a difference in complex formation between the sample compared to the control sample is indicative of the presence of CD47 in the sample.

Also within the scope of the invention are kits consisting of the compositions of the invention and instructions for use. The kit can further contain a least one additional reagent. Kits typically include a label indicating the intended use of the contents of the kit. The term label includes any writing, or recorded material supplied on or with the kit, or which otherwise accompanies the kit. The kit may further comprise tools for diagnosing whether a patent belongs to a group that will respond to a treatment, as defined above.

The invention having been fully described, it is further illustrated by the following examples and claims, which are illustrative and are not meant to be further limiting.

### BRIEF DESCRIPTION OF THE FIGURES

FIG. 1. Murine SIRPα-Fc binds CD47+/+ (WT) but not CD47−/− (KO) cells. Murine SIRPα Fc binding to CD47+/+ (WT) or CD47−/− (KO) murine splenocytes was detected by flow cytometry as described. The fluorescence resulting from SIRPα-Fc binding (SIRP) is plotted as dot plot versus FL3.

FIG. 2. Human SIRPα-Fc binds to CD47+/+ expressing (Jin8CD47) but not CD47 deficient Jurkat T cells (Jin8). SIRPα-Fc binding was quantified by flow cytometry as described. The fluorescence resulting from SIRPα-Fc binding (SIRP) is plotted as histogram in bold line. Bold line indicates binding in presence of 10 ug/mL anti CD47 clone B6H12.

FIG. 3. Blocking CD47/SIRPα at priming prevents allergic disease development in BALB/c mice, (3a) Mice were OVA-sensitized i.p. on day 0 and 5 in the presence or absence of SIRPα-Fc fusion molecules, and received OVA-aerosol challenges on day 12, 16 and 20 and euthanized on day 21 (n=4 to 7 mice per group). (3b) Lung sections of naïve (PBS). OVA immunized, OVA plus SIRP-α-Fc-treated mice stained with H&E and PAS. Data are representative of 3 individually analyzed lungs. (3c) Differential BALF cell numbers were analyzed by flow cytometry. (3d) Serum levels of OVA-specific IgE measured at day 21. (3e) IL-4, IL-5 and IL-13 levels in the supernatants of mLN cell cultures after 3 days of in vitro restimulation with OVA. (3f) At day 21, % of CD4+ T cells and IL-13 producing CD4+ T cells were assessed by flow cytometry in ex vivo isolated mLNs. Shown is one representative experiment out of 3. (3g) IL-4, IL-5, IL-13 and (h) eotaxin release in lung explants. Data are mean±SEM. *P<0.05, **P<0.01, ***P<0.001.

FIG. 4. Mechanisms of disease protection in SIRPα-Fc treated BALB/c mice, Mice were OVA-sensitized on day 0 and 5 in the presence or absence of SIRPα-Fc fusion molecule, received OVA-aerosol challenges on day 12, 16 and 20 and were euthanized on day 21. (4a) Subpopulations of CD11blowCD103+ and CD11 bhighCD103-DCs (gated on CD11c+) in mLNs. Data are presented as % DC subsets (n=3 to 4 mice per group). (4b) BALB/c mice were passively transferred with CFSE-labeled CD47+/+CD4+Tg T cells a day prior i.p. OVA-alum immunization in the absence (PBS) or presence of SIRPα-Fc and CFSE cell dilution was examined in mLNs after 2 days. Data is one representative experiments performed on 4 mice per group. (4c) At day 21, absolute numbers of eosinophils (CCR3+) were assessed by flow cytometry in ex vivo isolated mLNs. Data are mean±SEM (n=3 to 4 mice per group).

FIG. 5. Protection of TNBS-Colitis by SIRPα-Fc Administration

Colitis was induced and assessed as described. 100 ug/animal murine SIRPα-Fc was applied at day 0 and 24 h after i.p. Alternatively PBS was injected i.p. Bodyweight of animals was assessed until day 4 post TNBS injection.

### EXAMPLES

### 1. Examples of Soluble Polypeptides of the Invention

The following table 3 provides examples of Soluble Polypeptides of the Invention that may be produced by recombinant methods using DNA encoding the disclosed amino acid sequences.

TABLE 3

| | | SIRPα-derived | | Fc | Full |
|---|---|---|---|---|---|
| Example | Leader | polypeptide | Linker | sequence | SEQ ID |
| #1 | NO: 1 | NO: 2 | No linker | NO: 13 | NO: 14 |
| #2 | NO: 1 | NO: 2 | NO: 9 | NO: 13 | NO: 15 |
| #3 | NO: 1 | NO: 2 | NO: 12 | NO: 13 | NO: 16 |
| #4 | NO: 1 | NO: 3 | NO: 7 | NO: 13 | NO: 17 |
| #5 | NO: 1 | NO: 3 | NO: 8 | NO: 13 | NO: 18 |
| #8 | NO: 1 | NO: 5 | No linker | NO: 13 | NO: 19 |
| #7 | NO: 1 | NO: 5 | NO: 9 | NO: 13 | NO: 20 |
| #8 | NO: 1 | NO: 6 | NO: 7 | NO: 13 | NO: 21 |
| #9 | NO: 1 | NO: 6 | NO: 10 | NO: 13 | NO: 22 |

### 2. Affinity Determination

### 2.1 Affinity to CD47

The affinity of human SIRPα-Fc to monomeric CD47 or divalent CD47-Fc protein can be assessed by Biacore. Monovalent interaction of CD47 V-domain with SIRPα is reported around 1 μM (Heatherley et al Mol Cell, 2008).

For example, an APP-tagged CD47 V domain protein is expressed in HEK293 cells and compared to a divalent-CD47-Fc protein as ligand. The monovalent interaction with SIRPα-Fc was measured as K₀ of 0.8 μM while the binding strength (avidity) of a divalent CD47-Fc protein increased by a factor of 10 to K_{D}<60 nM. In contrast binding of a murine CD47-Fc fusion protein could not be observed indicating the specificity of the assessed interaction.

TABLE 4
Binding affinities determined by Biacore analysis

| | Mol weight | |
|---|---|---|
| | Da | K_{D} [uM] to SIRPα-Fc |
| mouse CD47-huFc (control) | 80805 | >10 (non detectable |
| | | binding) |
| huCD47-huFc (divalent) | 81317 | 0.06 |
| huCD47-APP (monovalent) | 16916 | 0.8 |

### 2.2 Cell Binding to Murine Cellular CD47

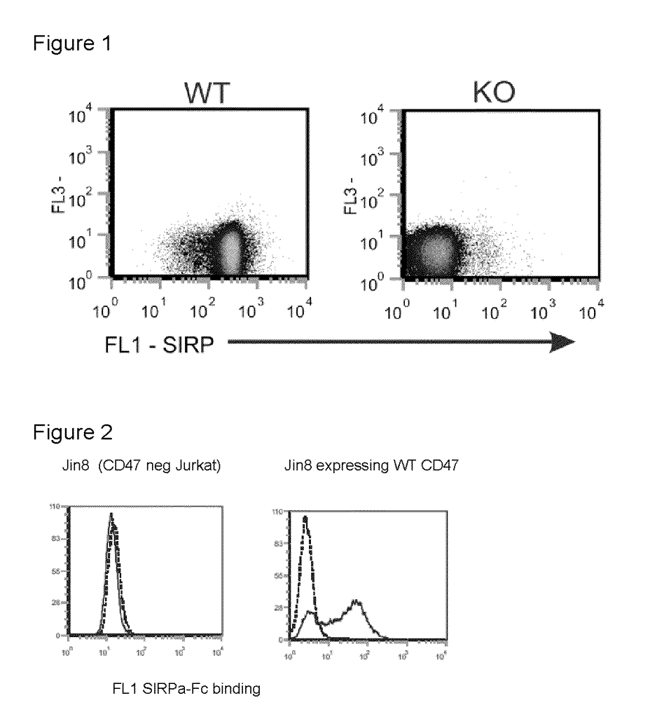

5×10⁵ CD47⁺ and CD47⁻ murine splenocytes from wild type or CD47 knock out mice are resuspended in 50 μl of FACS BUFFER (PBS 2% FCS 2 mM EDTA) containing 200 μg/ml human IgG and 5 μg/ml murine SIRPα-Fc for 30 min at 4° C. After washing, cells are stained with FITC-labeled streptavidin (1/1000) for 30 min at 4° C. The results show that SIRPα-Fc binds to lymphocytes of CD47^{+/+} but not to lymphocytes from CD47^{-/-} knockout mice (FIG. 1).

### 2.3 Cell Binding to Human Cellular CD47

5×10⁵CD47⁺ and CD4⁻ Jurkat T cell lines are resuspended in 50 μl of FACS BUFFER (PBS 2% FCS 2 mM EDTA) containing 200 g/ml human IgG and 5 μg/ml SIRPα-Fc for 30 min at 4° C. After washing, cells are stained with FITC-labeled streptavidin (1/1000) for 30 min at 4° C.

The results show that SIRPα-Fc binds to lymphocytes of CD47^{+/+} Jurkat cells (Jin8CD47) but not to Jurkat cells which do not express CD47 (Jin8) (FIG. 2). Binding to cells was specific as indicated by complete blockade with anti CD47 antibody B6H12.

### 2.4 Inhibition/Blocking Studies of Biotinylated SIRP-α Fc Binding to CHO CD47 Cell Lines

Alternatively, inhibition/blocking studies of biotinylated SIRPα-Fc binding to CHO CD47 cell lines may be performed using anti-CD47 mAbs directed against different epitopes (i.e. B6H12, 2D3, BRIC 126, IF7 and 10G2 clones, different ant-human SIRP-α (CD172a) mAbs, recombinant human Thrombospondin-1 (TSP-1) or TSP-1c-terminal (4N1K) and control (4NGG) peptides.

### 2.5 Blocking/Inhibition Studies of Directly Labeled Anti-CD47 mAb Binding to CHO CD47 Cell Lines

As a complementary approach, blocking/inhibition studies of directly labeled anti-0047 mAb binding to CHO CD47 cell lines may be performed using non biotinylated human SIRP-α-Fc.

Inhibition/blocking studies of biotinylated CD47-Fc binding to L cells transfected with human SIRPα-Fc using SIRPα-Fc can also be assessed.

### 3. SIRP-α-Fc Functional Assays

### 3.1 Immune Complex-Stimulated Dendritic Cell Cytokine Release Assay

Peripheral blood monocytes (C014+ CD16−) as well as monocyte-derived dendritic cells (DCs) are prepared as described (Latour et al, J of Immunol, 2001: 167:2547), Conventional (DCs) are isolated as CD11c+, lineage—, by a FACS Aria (BD Biosciences) by using allophycocyanin (APC)-labeled anti-CD11c (B-ly6), a mixture of FITC-labeled mAbs against lineage markers, CD3, CD14, CD15, CD16, CD19 and CD56 and APC-Cy7-labeled CD4 (RPA-T4) to reach >99% purity. APCs are stimulated with Staphylococcus Aureus Cowan 1 at 1/40,000 (Pansorbin) or soluble CD40L (1 μg/ml) and IFN-γ (500 U/ml) in the presence of various concentrations of human SIRPα-Fc (1 to 20 in HB101 serum-free medium. Cytokine (IL-1, IL-6, IL-10, IL-12p70, IL-23, IL-8 and TNF-α) release is assessed by ELISA in the 24 h or 48 h culture supernatants.

### 3.2 Mixed Lymphocyte Reactions (MLR) Assay

Mitomycin c-treated mature DCs (SAC or LPS stimulated) will be cocultured with allogeneic unfractionated, naïve (CD45RA⁺ CD62L^{High}) or memory (CC/45RO⁺ CD62L^{low}) adult CD4+ T cells (10⁶/ml) at various stimulators (DCs)/responder (T cells) ratios in the presence or absence of Soluble Polypeptides of the Invention (5 to 50 μg/ml). T cell proliferation (³H thymidine uptake) and IFN-γ release will be assessed in the culture supernatants of 5 to 6 day primary cultures.

### 4. In Vivo Data with Murine Animal Models for Use of SIRPα-Fc in the Treatment of Asthma

BALB/c mice were sensitized by i.p. injection of 10 μg OVA (Sigma, Grade V) adsorbed to 1 mg Imject Alum (Pierce) on days 0 and 5. On days 12, 16 and 20, mice were challenged for 30 minutes with a 0.5% OVA aerosol (Sigma, Grade V) delivered by a vibrating mesh nebulizer system (Omron). 24 hours after the last challenge, mice were sacrificed with 75 mg/kg sodium pentobarbital overdose and bled. BAL was collected 3 times with 0.5 ml physiologic saline and lung and mLN were isolated. One third of the lungs were rinsed in PBS supplemented with antibiotics, cut into small pieces and put in culture in flat-bottom 24 well plates for 24 h in 1 ml RPMI1640 (Wisent Inc.) supplemented with 10% fetal bovine serum, 500 U/ml Penicillin, 500 μg/ml Streptomycin, 10 mM HEPES buffer and 1 mM 2-ME. MLN cells (4×106 cells/ml) were cultured in flat-bottom 96 well plates and restimulated with OVA (100 μ/ml) for 72 h. Total BAL cells were washed, counted and stained for 30 minutes with anti-CCR3PE (R&D systems), anti-CD3 FITC (clone 145-2C11) and anti-B220 FITC (R&D systems). As described in Van Rijt L. S. et al (Immunol Methods, 2004 May; 288(1-2):111-21), granulocytes were found to be granular, non-autofluorescent, lacking the expression of CD3 and B220. Eosinophils were distinguished from neutrophils by CCR3 expression. Lymphocytes were small, non-granular, non-autofluorescent, expressing CD3 or B220 and the other mononuclear cells, including macrophages and DCs, were lacking CD3, B220 and CCR3. To identify the DC subsets, mLNs and lungs were first treated with liberase, minced and cells were counted, Lung cell suspensions were treated with NH4Cl for red blood cell lysis and washed before staining. Cells were stained with anti-CD11c FITC (BD Biosciences) or anti-CD11c APC (clone N418), anti-CD11b PE (Caltag), anti-1-Ad/1-Ed PE (BD Biosciences), anti-GR1, anti-B220 FITC (R&D systems), 120G8 FITC and anti-CD103-biotinylated followed by SA-APC or CD103 PE (BD Biosciences) anti-CD47 and anti-SIRP-α mAbs. In lungs, autofluorescent alveolar macrophages were excluded from the analysis gates. To identify regulatory T cells, anti-CD4 FITC or APC (BD Biosciences), anti-CD25 PE (Caltag) or FITC (BD Biosciences), anti-CD44 APC (clone IM7 8.1) were used. To measure IL-13 production ex vivo, mast cells and basophils were first identified with extracellular anti-IgE-biotinylated and anti-CD117 (c-Kit, BioLegend) and CD4 T cells were stained with anti-CD4 APC. Cells were fixed, permeabilized and stained with anti-IL-13 PE (eBioscience). Cells were first stained for extracellular markers (anti-CD4 APC and anti-CD25 FITC), fixed, permeabilized and stained with anti-FoxP3 PE (kit from eBioscience). Mast cells and basophils+Intracytoplasmic IL-13 staining. All the data were acquired on a FACSCalibur or Cantoll Row Cytometer (BD Biosciences) and analysed with Cellquest or DIVA software (BD Biosciences).

### Cytokine Measurements

IL-2, IL-4, IL-5, IL-10, IFN-γ (BD Biosciences), IL-13 (R&D Systems) are measured in mLNs culture supernatants and lung explants by ELISA.

Mediastinal LN cells from OVA-sensitized and challenged mice are restimulated in vitro for 3 days with OVA protein (1 mg/ml) and IL-4, IL-5 and IL-13 production are quantified by ELISA in the culture supernatants. Lungs explants are cultured overnight in complete medium and culture supernatants are collected to measure cytokine release.

### Results

In Vivo Data with Murine Animal Models for Use of SIRPα-Fc in the Treatment of Allergic Asthma

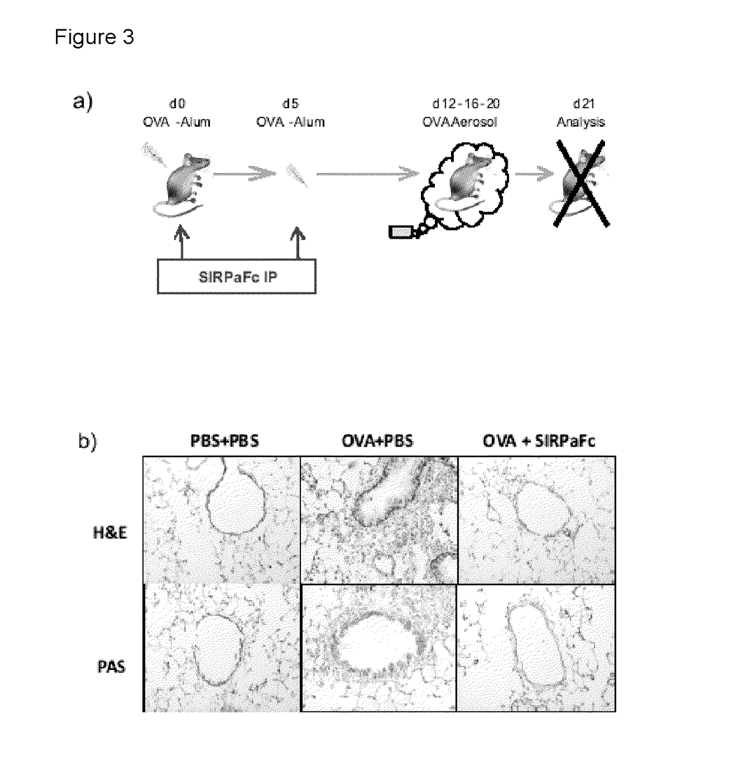

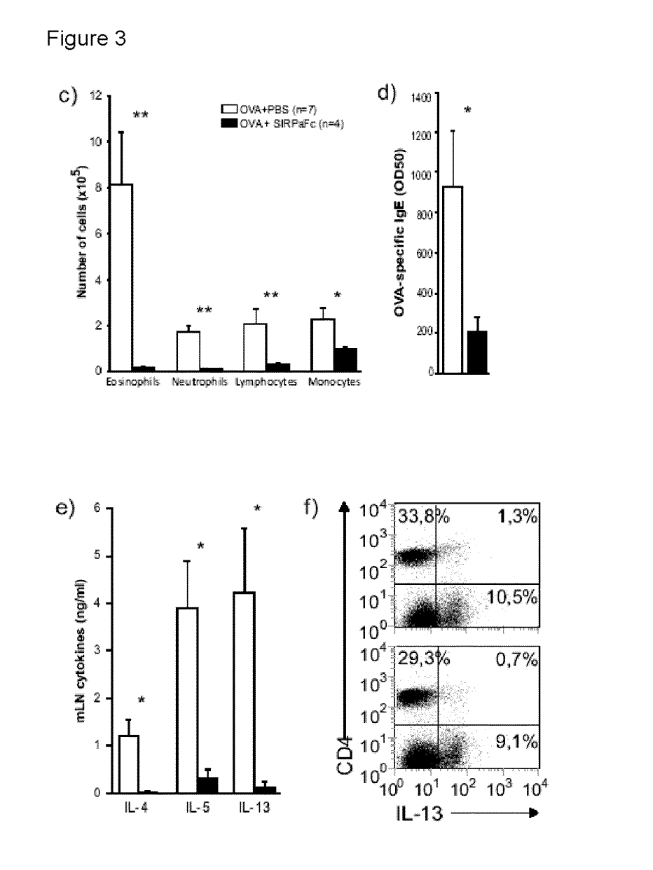

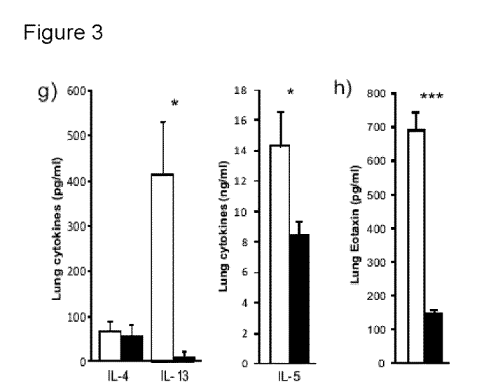

CD47 and SIRPα appear as important molecules in the initiation and perpetuation of SIRP-α+CD103− DC-driven Th2 immunity. Thus they may therefore be harnessed therapeutically to reduce lung inflammation and ameliorate airway disease. We here assessed the efficacy of SIRP-α plus the Fc region of human IgG1 (SIRPα-Fc) on the development of allergic airway inflammation. BALB/c mice administered either SIRPα-Fc on days 0 and 5 of OVA immunization (FIG. 3a) had very few or no inflammatory cell infiltration of the lung tissues after OVA aerosol challenge (FIG. 3b). A strong reduction or an absence of eosinophils, neutrophils and lymphocytes in the BALF (FIG. 3c) occurred together with a drop in serum OVA-specific IgE (FIG. 3d), a 50% reduction in lymph node cellularity and a drastic inhibition of IL-4, IL-5 and IL-13 production in the mLNs (FIGS. 3e and f). The protection from airway disease development was not correlated with an increase in IL-10 nor IFN-γ release, which in fact was also suppressed in treated mice (data not shown). We next examined cytokine and chemokine release in the culture supernatants of lung explants of CD47− and SIRPα-Fc-treated mice and found that IL-5, IL-13 and eotaxin release were inhibited while IL-4 expression remained unchanged (FIGS. 3g and h).

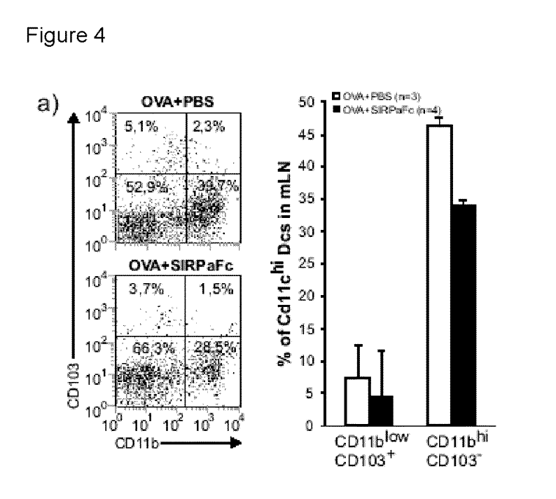

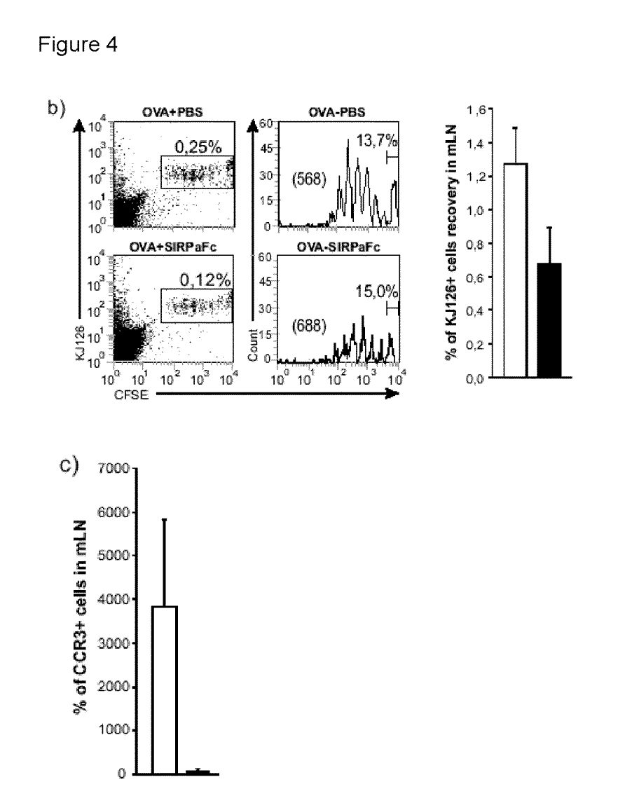

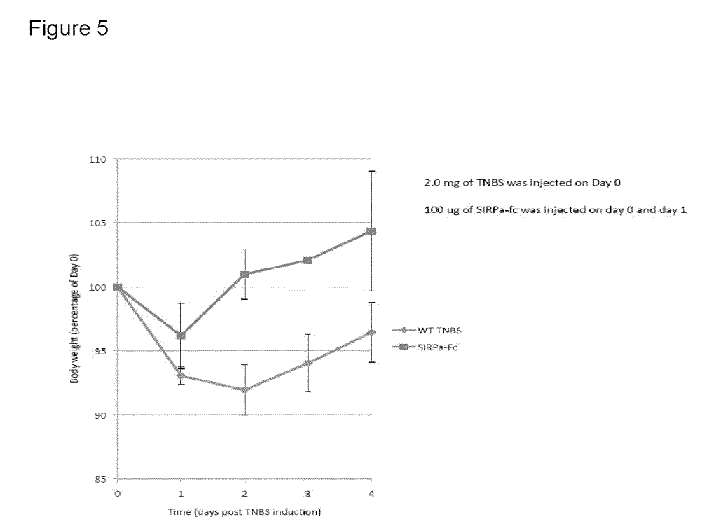

We next explored the potential mechanisms that governed this inhibition and resulted in protection from airway disease. We found a diminution in the accumulation of SIRP-α+CD103− DCs in the mLNs of CD47-Fc-treated mice (FIG. 4a). The administration of SIRPα-Fc also led to a reduction in the proportion of CFSE-labeled Tg T cells in the mLNs of OVA-immunized mice treated with CD47-Fc (FIG. 4b). Finally, we observed a decrease in the proportion and accumulation of eosinophils in the mLns of SIRPα-Fc-treated mice (FIG. 4c).

These data demonstrate that CD47/SIRPα interruption at primary Ag sensitization drastically reduced type 2 responses in mLNs and lungs as well as IgE-dependent airway inflammation.

### 5. In Vivo Data with Murine Animal Models for Use of SIRPα-Fc in the Treatment of Colitis

Trinitrobenzene sulfonic acid (TNBS) (2 or 3 mg) was dissolved in 50% ethanol and instilled into the colons of male Balb/c mice (WT and CD47 KO) via a 3.5F catheter. Control mice were given ethanol alone. Mice were weighed every 24 hours and sacrificed on day 2 (early time point) or day 4. In the chronic TNBS colitis model, 1.5 mg of TNBS was instilled intrarectally on day 0 and again on day 7, and mice were sacrificed on day 12. Serum, mesenteric lymph nodes and colons were harvested for further analysis. Colons were scored macroscopically using the Wallace criteria which takes into account the presence of diarrhea, adhesions, thickening of the bowel wall and ulceration. They were also evaluated for microscopic markers of inflammation using the Ameho criteria, a scoring system based upon thickening of the submucosa, infiltration of the submucosa and lamina propria with mononuclear cells, mucous depletion, loss of crypt architecture, and edema (not shown). A recombinant mouse SIRPα-Fc fusion protein was administered intraperitoneally (100 ug/mouse) just prior to TNBS colitis induction and 24 hours thereafter. Control mice received saline alone. Injection of murine SIRPα-Fc 100 μg/animal on day 0, 30 min before TNBS induction and on day 1 blocked statistically significantly the disease development as assessed by bodyweight loss.

### 6 In Vivo Murine Animal Models for Use of SIRPα-Fc in the Treatment of Arthritis

### Collagen Induced Arthritis Model

Mycobacterium tuberculosis are mixed with Freund's complete adjuvant and thoroughly shaken (=solution A). Bovine collagen solution aliquots are well mixed on ice with sterile PBS (=solution B). Solution A and solution B were injected as emulsion into naïve male DBA/1 mice. The mice are anaesthetized by s.c. injection of a sterile filtrated mixture of ketamine. Upon narcosis, the root of the tail of each mouse is shaved and subsequently. 0.1 ml of the collagen-emulsion per mouse (containing 100 μg of collagen) is injected i.d. into the base of the tail. A second injection of 100 μl of collagen/PBS (1: dilution) is given i.p. on day 22 after the first immunization (=booster). Swelling and scoring of disease is assessed as described in Nat. Protoc. 2007; 2(5):1269-75 by Brand et al.

### 7. Useful Amino Acid and Nucleotide Sequences for Practicing the Invention

TABLE 3

| SEQ ID | SEQUENCE |
|---|---|
| SEQ ID NO: 1 | Wild-type Sirpα leader sequence |
| SEQ ID NO: 2 | Wild-type Sirpα D1 domain sequence |
| SEQ ID NO: 3 | Wild-type Sirpα D1-D2-D3 domain sequence |
| SEQ ID NO: 4 | Sirpα D1 domain polymorphic sequence T50S |
| SEQ ID NO: 5 | Sirpα D1 domain polymorphic sequence D95E |
| SEQ ID NO: 6 | Wild-type Sirpα D1-D2-D3 domain polymorphic |
| | sequence V302L |
| SEQ ID NO: 7 | Wild type FC linker sequence from IgG1 |
| SEQ ID NO: 8 | FC linker mutated seauence from IgG1 |
| | (Cys replaced by Ser) |
| SEQ ID NO: 9 | Wild type FC linker sequence from IgG1 |
| SEQ ID NO: 10 | FC linker mutated sequence from IgG1 |
| | (Cys replaced by Ser) |
| SEQ ID NO: 11 | No wild-type linker sequence |
| SEQ ID NO: 12 | No wild-type linker sequence |
| SEQ ID NO: 13 | IgG1 silent FC sequence |
| SEQ ID NO: 14 | SEQ ID: 1 + SEQ ID: 2 + SEQ ID: 13 |
| SEQ ID NO: 15 | SEQ ID: 1 + SEQ ID: 2 + SEQ ID: 9 + SEQ ID: 13 |
| SEQ ID NO: 16 | SEQ ID: 1 + SEQ ID: 2 + SEQ ID: 12 + SEQ ID: 13 |
| SEQ ID NO: 17 | SEQ ID: 1 + SEQ ID: 3 + SEQ ID: 7 + SEQ ID: 13 |
| SEQ ID NO: 18 | SEQ ID: 1 + SEQ ID: 3 + SEQ ID: 8 + SEQ ID: 13 |
| SEQ ID NO: 19 | SEQ ID: 1 + SEQ ID: 5 + SEQ ID: 13 |
| SEQ ID NO: 20 | SEQ ID: 1 + SEQ ID: 5 + SEQ ID: 9 + SEQ ID: 13 |
| SEQ ID NO: 21 | SEQ ID: 1 + SEQ ID: 6 + SEQ ID: 7 + SEQ ID: 13 |
| SEQ ID NO: 22 | SEQ ID: 1 + SEQ ID: 6 + SEQ ID: 10 + SEQ ID: 13 |
| SEQ ID NO: 23 | Full length human SIRPα (NP_542970.1) |
| SEQ ID NO: 24 | Human CD47(NP_001768.1) |
| SEQ ID NO: 25 | DNA coding sequence of SEQ ID NO: 1 |
| SEQ ID NO: 26 | DNA coding sequence of SEQ ID NO: 2 |
| SEQ ID NO: 27 | DNA coding sequence of SEQ ID NO: 3 |
| SEQ ID NO: 28 | DNA coding sequence of SEQ ID NO: 4 |
| SEQ ID NO: 29 | DNA coding sequence of SEQ ID NO: 5 |
| SEQ ID NO: 30 | DNA coding sequence of SEQ ID NO: 6 |

1. A soluble CD47 binding polypeptide for use as a medicament, comprising a SIRPα-derived polypeptide selected among the group consisting of:
a) an extracellular domain of SIRPα (SEQ ID NO:3);
b) a fragment of SEQ ID NO:3, and,
c) a variant polypeptide of SEQ ID NO:3 having at least 75% identity to SEQ ID NO:2;
wherein said SIRPα-derived polypeptide binds to human CD47 (SEQ ID NO:24).

2. The soluble CD47 binding polypeptide according to claim 1, wherein said soluble CD47 binding polypeptide binds to human CD47 with a K_{D} of 2 μM or less, and inhibits induced cytokine secretion as measured in an immune complex-stimulated dendritic cell cytokine release assay.

3. The soluble CD47 binding polypeptide according to claim 1, wherein said SIRPα derived polypeptide is fused to an IgG Fc fragment.

4. The soluble CD47 binding polypeptide according to claim 1, wherein said IgG Fc fragment is a mutant aglycosylated Fc fragment.

5. The soluble CD47 binding polypeptide according to claim 1, wherein said extracellular domain of SIRPα comprises at least the V-region of SIRPα (SEQ ID NO:2).

6. (canceled)

7. (canceled)

8. The soluble CD47 binding polypeptide according to claim 1, wherein said polypeptide essentially consists of an extracellular domain of SIRPα fused to the Fc fragment of a human IgG.

9. A protein comprising at least two soluble CD47 binding polypeptides according to claim 1.

10. A pharmaceutical composition comprising the soluble CD47 binding polypeptide according to claim 1.

11. The pharmaceutical composition of claim 10, in combination with one or more of a pharmaceutically acceptable excipient, diluent or carrier.

12. An isolated nucleic acid comprising the sequence encoding the soluble CD47 binding polypeptide according to claim 1.

13. A cloning or expression vector comprising one or more nucleic acids according to claim 12.

14. A cloning or expression vector according to claim 13, comprising at least one nucleic acid of SEQ ID NO: 25 or 26.

15. A host cell comprising one or more cloning or expression vectors according to claim 13.

16. The host cell according to claim 15, wherein said host cell is a mammalian cell, for example, CHO cell.

17. A pharmaceutical composition comprising a protein comprising at least two soluble CD47 binding polypeptides according to claim 1.

18. A method of treating an autoimmune or inflammatory disorders, comprising administering to a subject an effective amount of the soluble CD47 binding polypeptide according to claim 1.

19. A method of treating an autoimmune or inflammatory disorders, comprising administering to a subject an effective amount of the soluble CD47 binding polypeptide according to claim 17.

20. The method of claim 18 wherein the autoimmune or inflammatory disorder is:
a) Th2-mediated airway inflammation;
b) allergic disorders;
c) asthma;
d) inflammatory bowel diseases;
e) arthritis;
f) ischemic disorders; or,
g) leukemias or cancer.
